(19) United States
(12) Patent Application Publication
(10) Pub. No.: US 2023/0332200 A1
(43) Pub. Date: Oct. 19, 2023

(54) A PROCESS FOR THE PREPARATION OF SEMAGLUTIDE AND SEMAPEPTIDE

(71) Applicant: DR. REDDY'S LABORATORIES LIMITED, Hyderabad (IN)

(72) Inventors: Manpreet SINGH, Barnala (IN); Jyothishwaran GANESHAN, Hyderabad (IN); Prasad ERNALA, Nizamabad District (IN); Krutik DOSHI, Hyderabad (IN); Manoj Kumar SINGH, Hyderabad (IN); Goutham KUMAR, Hyderabad (IN); Ramgopal KANDELA, Gadwal (IN); Somasekharan SANIL, Hyderabad (IN); Rajeev Rehani BUDHDEV, Hyderabad (IN)

(21) Appl. No.: 18/026,935

(22) PCT Filed: Sep. 21, 2021
(86) PCT No.: PCT/IN2021/050924

§ 371 Date: Mar. 17, 2023

(30) Foreign Application Priority Data
Sep. 23, 2020 (IN) 202041041229

(51) Int. Cl. C12P 21/02; C12N 15/70; C07K 14/605

(52) U.S. Cl. CPC C12P 21/02; C12N 15/70; C07K 14/605; C07K 2319/20; C12N 2310/3517

(57) ABSTRACT

The present application relates to a process for the preparation of semaglutide. The present application also relates to a recombinant process for the preparation of semapeptide. The present invention is related to a process for producing semapeptide, the process comprising the steps of, a) culturing a host cell comprising a nucleotide sequence encoding of Formula (II) under suitable conditions for expression, wherein, insoluble tag is a nucleotide sequence of Alanine-Valine; b) recovering semapeptide, wherein semapeptide amino acid sequence is Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly.

### FIELD OF THE INVENTION

The present application relates to a process for the preparation of semaglutide. The present application also relates to a recombinant process for the preparation of semapeptide.

### BACKGROUND OF THE INVENTION AND DISCLOSURE OF PRIOR ART

Semaglutide is a GLP-1 analogue with 94% sequence homology to human GLP-1. GLP-1 is a physiological hormone that has multiple actions on glucose, mediated by the GLP-1 receptors. Semaglutide, proprietary name OZEMPIC®, developed by Novo Nordisk and first approved by USFDA on 5 Dec. 2017 as a GLP-1 receptor agonist and indicated as an adjunct to diet and exercise to improve glycemic control in adults with type 2 diabetes mellitus. OZEMPIC®, 2 mg/1.5 mL (1.34 mg/mL) injection for subcutaneous use is available in:(i) Single-patient-use pen that delivers 0.25 mg or 0.5 mg per injection, & (ii) Single-patient-use pen that delivers 1 mg per injection. Semaglutide, proprietary name RYBELSUS®, developed by Novo Nordisk was first approved by USFDA on 20 Sep 2019 as a GLP-1 receptor agonist for oral use in strength 3 mg, 7 mg and 14 mg.

Semaglutide is chemically known as N-□²⁶-[2-(2-[2-(2-[2-(2-[4-(17-Carboxyhepta-decanoylamino)-4(S)-carboxybutyrylamino]ethoxy)ethoxy]acetylamino)ethoxy]ethoxy)acetyl][Aib⁸,Arg³⁴]GLP-1-(7-37)peptide. The molecular formula is C₁₈₇H₂₉₁N₄₅O₅₉ and the molecular weight is 4113.58 g/mol. Semaglutide can be represented by the following structural Formula I:

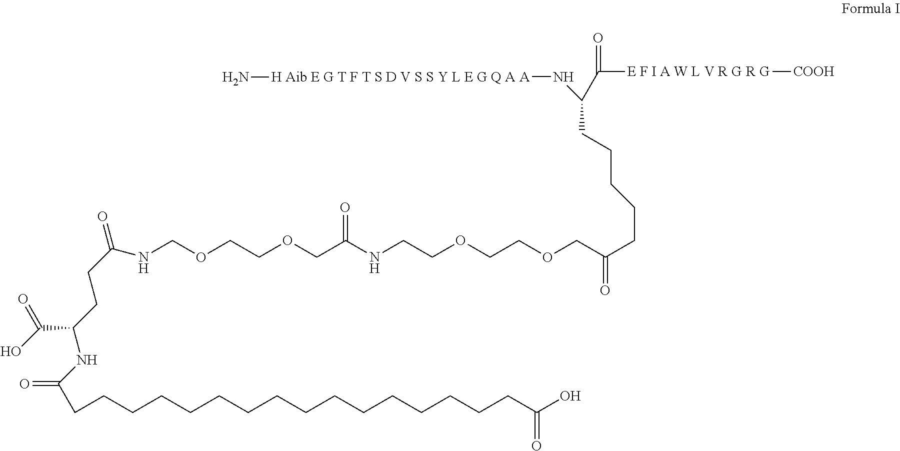

U.S. Pat. No. 8,129,343B2 reported solid phase peptide synthesis of Semaglutide. Other patents/applications such as U.S. Pat. Nos. 8,637,647, 9,732,137, WO2013098191A1, WO2016046753A1, WO2018032843A1, WO2017114191A1, WO2019170895A1, WO2019170918A1, WO2019120639A1 and WO2020074583A2 reported the preparation of semaglutide via fermentation, solid phase peptide synthesis or fragmentation approaches.

U.S. Pat. No. 9,732,137, discloses the preparation of semapeptide by using yeast as a host cell. CN110498849A also discloses the preparation of semapeptide by using a synthetic construct of “leader peptide-DDDDK-GLP-1 (9-37)” in E. Coli.

Even though, the abovementioned prior arts disclose processes for the preparation of semaglutide via semi-recombinant methods as well as synthetic methods, there is still a need for an improved process for enabling the industrial production of semaglutide.

The objective of the present application is to provide an improved recombinant process for the preparation of semapeptide.

All references cited herein are incorporated by reference in their entireties for all purposes.

### SUMMARY OF THE INVENTION

In the first embodiment, the present invention is related to a process for the preparation of semaglutide.

In the second embodiment, the present invention is related to a process for producing semapeptide, the process comprising the steps of:
a) culturing a host cell comprising a nucleotide sequence encoding of Formula (II) under suitable conditions for expression,

Insoluble tag-Fusion tag-Semapeptide (Nucleotide Sequence) Formula (II)

wherein, insoluble tag is a nucleotide sequence of Alanine-Valine;
b) recovering semapeptide, wherein semapeptide amino acid sequence is Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly.

In an aspect of second embodiment, the said insoluble tag is optionally linked to a nucleotide sequence encoding affinity tag to give Formula (III),

Affinity Tag-Insoluble tag-Fusion tag-Semapeptide (Nucleotide Sequence) Formula (III)

In the third embodiment, the present invention is related to increase in accumulation of resulting semapeptide by fermentation process.

In fourth embodiment, the present invention is related to a process for the preparation of semaglutide, comprising the steps of:
a) culturing a host cell comprising a nucleotide sequence encoding of Formula (II) or Formula (III) under suitable conditions for expression,

Insoluble tag-Fusion tag-Semapeptide (Nucleotide Sequence) Formula (II)

Affinity Tag-Insoluble tag-Fusion tag-Semapeptide (Nucleotide Sequence) Formula (III)

wherein, insoluble tag is nucleotide sequence of Alanine-Valine;
b) recovering, semapeptide, wherein semapeptide amino acid sequence is Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly;
c) coupling, optionally protected Aib and His amino acid,
d) acylating an epsilon amino group of lysine residue in semapeptide with an acylating agent of Formula (V), which is optionally activated,

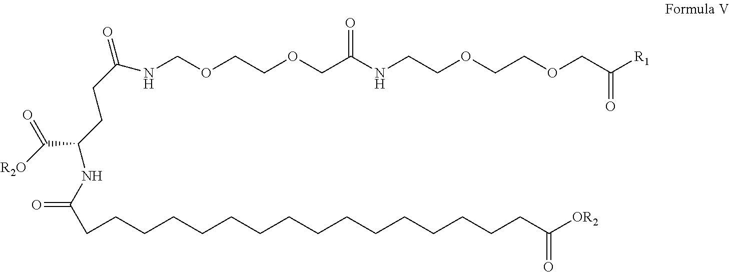

wherein, R₁ is hydroxy or a reactive ester thereof, R2 is selected from H or C₁₋₁₂ alkyl;
e) isolating semaglutide.

### BRIEF DESCRIPTION OF THE DRAWINGS

FIG. 1: Recombinant plasmid of pET24-His6-AV-Ubiquitin-Semapeptide.

FIG. 2: PCR Amplification of His6-AV-Ubiquitin-Semapeptide Gene.

FIG. 3: Colony PCR of E.Coli Top10-pET24-His6-AV-Ubiquitin-Semapeptide Gene (clones) using T7 Primers.

FIG. 4: Restriction digestion analysis of pET24-His6-AV-Ubiquitin-Semapeptide Gene (clones).

FIG. 5: Screening of E.coli JM109DE3-pET24-His6-AV-Ubiquitin-Semapeptide Expression Gene (clones) for the His6-AV-Ubiquitin-Semapeptide Expression.

FIG. 6: Solubility Test of E.coli JM109DE3-pET24-His6-AV-Ubiquitin-Semapeptide Gene (clones).

### DETAILED DESCRIPTION

In the first embodiment, the present invention is related to a process for the preparation of semaglutide.

In an aspect of first embodiment, the present invention is related to the preparation of semaglutide from semapeptide.

In another aspect of first embodiment, the present invention is related to a process for the preparation of semaglutide from recombinantly produced semapeptide.

In the second embodiment, the present invention is related to a process for producing semapeptide, the process comprising the steps of:
a) culturing a host cell comprising a nucleotide sequence encoding of Formula (II) under suitable conditions for expression,

Insoluble tag-Fusion tag-Semapeptide (Nucleotide Sequence) Formula (II)

wherein, insoluble tag is a nucleotide sequence of Alanine-Valine;
b) recovering semapeptide, wherein semapeptide amino acid sequence is Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly.

In an aspect of second embodiment, present invention involves the preparation of fusion tag construct of Formula (II) comprising fusion of amino terminal of a synthetic nucleotide of semapeptide with carboxyl terminal of fusion tag construct. According to present invention, the fusion tag construct further fused with insoluble tag.

In an aspect of second embodiment, the said insoluble tag is optionally linked to a nucleotide sequence encoding affinity tag to give Formula (III),

Affinity Tag-Insoluble tag-Fusion tag-Semapeptide (Nucleotide Sequence) Formula (III)

In another aspect of second embodiment, present invention involves the preparation of fusion tag construct of Formula (III) comprising fusion of amino terminal of synthetic nucleotide of semapeptide with carboxyl terminal of fusion tag construct. According to present invention, the fusion tag construct operably connected with insoluble tag and affinity tag.

In a preferred embodiment, a nucleotide sequence encoding of Formula (III) is selected from following formula (IV):

His6 Tag-AV-Ubiquitin-Semapeptide (Nucleotide Sequence) Formula (IV)

In an aspect of the second embodiment, the process for semapeptide further comprises:
a) ligating a nucleotide sequence encoding of Formula (II) or Formula (III) in expression vector;
b) transforming said expression vector into host cell and inducing the expression to obtain expressed semapeptide.

According to the step a), the expression vector may already have desired restriction sites for ligation or can be introduced into expression vector by using restriction enzyme.

According to the step b), expression in recombinant expression host cell is induced by chemical agent. The suitable chemical agent used for induction can be selected from IPTG (isopropylthiogalactoside), tryptophan, nalidexic acid, oxalinic acid, nitrogen or sugars analogs; wherein sugar analogs can be selected from lactose, maltose, arabinose and the like.

The suitable affinity tag can be selected from Polyarginine-tag (Arg-tag), Polyhistidine-tag (His-tag), S-tag, SBP-tag (streptavidin-binding peptide), Maltose binding protein, chitin binding domain (CBD), GST tag (Glutathione S Transferase), CBP-tag (Calmodulin binding protein), and the like.

The suitable fusion tag can be selected from ubiquitin tag (wild or mutant), Sumo Tag, Thioredoxin Tag, Green fluorescent protein Tag, Fc Tag, Carbohydrate recognition domain tag and the like.

The suitable host cell can be selected from prokaryotic host cells or eukaryotic host cells. The suitable prokaryotic host cell used in cloning can be selected from E. coli, Pseudomonas flurescence, Bacillus subtitis and the like. The suitable E. coli strains used in expression can be selected from E. coli BL21(DE3) pLysS, E. coli JM109, E. coli JM109 (DE3), Rosetta, Origami and the like.

The transformation method can be selected from heat shock method, electroporation and the like.

In one variant, heat shock method involves heat shock to cells at about 42° C. for about 30 sec to 120 sec and subsequently kept on ice for about 2-10 minutes.

In an aspect, expression vector that is used in the process of the present invention can be commercially available expression vectors or custom designed vectors selected from pET vectors, pd451sR and the like. pET vectors may be selected from pET24a, pET28a or any other pET vector known to person skill in the art.

In an aspect of the present invention, expression can be carried out at a pH of about 5.0 to about 7.5 and/or at a temperature of about 18° C. to about 42° C.

The expression vector according to present invention comprises of promoters, selection marker, multiple cloning regions, origin of replication & operator /repressor system. The suitable promoter can be selected from T7, TRC, TRP, BAD, LacUV5 or their derivatives or combination thereof.

The suitable selection marker may be selected from kanamycin, ampicillin, chloramphenicol or tetracycline or their combinations in their wild or mutated forms. The suitable origin of replication can be selected from pUC ORI, pPR322 and the like in their wild type or mutated form.

The suitable Operator/repressor systems can be selected from Lac operon system (see Miller et al. “The operon”, Cold Spring Harbor Laboratory, 1980 and Hillen et al, J. Mol. Biol. 172, 185-201 [1984]).

In the third embodiment, the present invention is related to increase in accumulation of resulting semapeptide by fermentation process.

In an aspect of third embodiment, the fermentation process comprising the steps of:
a) inducing transformant prokaryotic cells which comprise of expression vector and a nucleotide sequence encoding of Formula (II) or Formula (III) in fermentation culture medium;
b) culturing the transformant prokaryotic cells under condition suitable for accumulation of expressed semapeptide;
c) recovering the expressed semapeptide;
d) optionally, purifying the expressed semapeptide;
e) enzymatically cleaving the fusion tag from the expressed semapeptide; and
f) recovering semapeptide;
g) optionally, purifying semapeptide, wherein semapeptide amino acid sequence is Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly.

In an aspect of third embodiment, present invention involves fermentation process for increase in accumulation of expressed semapeptide by inducing transformant prokaryotic cells in culture medium with chemical agent.

The fermentation may be carried out in fed-batch to produce expressed semapeptide. Improved expression of the present invention depends on various parameters of the fermentation process.

The suitable condition for accumulation of expressed semapeptide dependent on parameters like fermentation media, concentration of the inducer, feed media and nutrient feed rate. The fermentation medium is the medium required for the growth and expression of transformant prokaryotic cells at fermenter scale. Typically, the fermentation medium comprises of suitable salts, vitamins, carbon source and nitrogen source. The suitable salts can be selected from ammonium chloride, potassium dihydrogen phosphate, disodium hydrogen phosphate, sodium chloride, calcium chloride, magnesium chloride, EDTA sodium salt, sodium molybdate, zinc sulphate, ferrous sulphate, copper sulphate, monopotassium phosphate, dipotassium phosphate, magnesium sulphate and the like or combination thereof. The carbon source may comprise glucose, glycerol, maltose, sucrose, dextrose, fructose or mannitol and the like or combination thereof The nitrogen source may comprise ammonia, nitrate, peptone, soya peptone, yeast extract, tryptone and the like or combination thereof The suitable vitamin can be selected from Thiamine (vitamin B or its related compounds) and the like or combination thereof. The fermentation medium further comprises acids selected from citric acid, boric acid and the like or combination thereof. The feed medium comprises of salt, carbon source, nitrogen source antibiotics and trace elements. The suitable salt, carbon source and nitrogen source are the same as defined herein above. The feed medium may comprise of antibiotics selected from kanamycin, ampicillin, chloramphenicol, tetracycline and the like and will depend upon the antibiotic marker gene embedded in the vector.

The expressed semapeptide accumulated in the cytoplasm of cells can be recovered/ harvested by conventional bacterial cell lysis techniques. In preferred embodiment of present invention, the expressed semapeptide refers to peptide sequence of His6-AV-Ubiquitin-semapeptide.

An aspect of present invention involves preparation of inclusion body. The inclusion body preparation involves resuspending of cell pellet in non-denaturing lysis buffer (Tris-HCl+Urea) by stirring and treating with lysozyme at room temperature. The resuspended cells can be homogenized under chilled conditions and centrifuged (Sorvall,Thermofisher). The unbroken cells, large cellular debris, and the inclusion body will be pelleted down and supernatant can be transfer from the pellet. The proteinaceous and non-proteinaceous contaminants present with inclusion body can be removed by washings. The pellet can be resuspended in wash buffer containing detergents selected from but not limited to sodium deoxycholate, Triton and Tween. The suspension can be centrifuged (Sorvall, Thermofisher).

In an aspect of present invention expressed semapeptide may be purified by the purification techniques selected from affinity chromatography i.e Ni NTA chromatography, ion exchange chromatography (cation or anion), reverse phase chromatography or any other technique well known in the art.

In an aspect of the present invention, the expressed semapeptide can be cleaved enzymatically in vivo or in vitro (using either pure or partially purified fusion specific protease) by cleavage enzyme which cleaves at the junction between ubiquitin fusion tag and semapeptide to generate the semapeptide. The cleavage enzyme can be selected from Yeast ubiquitin hydrolase (YUH1) and the like. In an aspect of the present invention, expression during fermentation can be carried out at a pH of about 5.0 to about 7.5 and/or at a temperature of about 25° C. to about 42° C.

In an aspect of present invention, purification of semapeptide is carried out by precipitation at its isoelectric (pI) point. In an aspect of the present invention, semapeptide isolated at isoelectric point has a purity of about 80% or about 90% or about 95% as measured by High Performance Liquid Chromatography (HPLC).

In fourth embodiment, the present invention is related to a process for the preparation of semaglutide, comprising the steps of:
a) culturing a host cell comprising a nucleotide sequence encoding of Formula (II) or Formula (III) under suitable conditions for expression,

Insoluble tag-Fusion tag-Semapeptide (Nucleotide Sequence) Formula (II)

Affinity Tag-Insoluble tag-Fusion tag-Semapeptide (Nucleotide Sequence) Formula (III)

wherein, insoluble tag is nucleotide sequence of Alanine-Valine;
b) recovering, semapeptide, wherein semapeptide amino acid sequence is Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly;
c) coupling, optionally protected Aib and His amino acid,
d) acylating an epsilon amino group of lysine residue in semapeptide with an acylating agent of Formula (V), which is optionally activated,

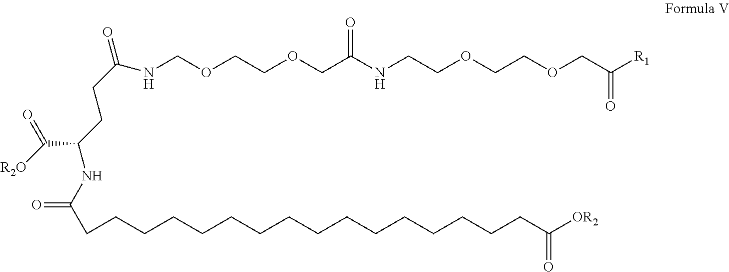

wherein, R₁ is hydroxy or a reactive ester thereof, R₂ is selected from H or C₁₋₁₂alkyl;
e) isolating semaglutide

In an aspect of fourth embodiment, optionally protected Aib is selected from Fmoc-Aib-OH and optionally protected His amino acid is selected from Boc-His(Boc)-OH.

DEFINITIONS The following definitions can be used in connection with the words or phrases used in the present application unless the context indicates otherwise.

The term “amino acid” as used herein refers to an organic compound comprising at least one amino group and at least one acidic group. The amino acid may be a naturally occurring amino acid or be of synthetic origin, or an amino acid derivative or amino acid analog. Amino acids may be referred to herein by either their commonly known three letter symbols or by the one-letter symbols recommended by the IUPAC-IUB Biochemical Nomenclature Commission. Likewise, nucleotides may be referred to by their commonly accepted single-letter codes.

The term “amplification” as used herein refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold 25 Spring Harbor Press, Plainview, N.Y.).

The term “peptide” as used herein refers to any peptide comprising two or more amino acid residues connected by peptide linkage.

The term “acylating” as used herein refers to the introduction of one or more acyl groups covalently bonded to the free amino groups of the protein or peptide. The term “acylation” means the acylation of the amino group of the protein or peptide.

The term “Semapeptide” or “semapeptide” is Arg34GLP-1 (9-37) peptide. The amino acid sequence of Semapeptide is: “Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly”

The term “semapeptide (Nucleotide sequence)” or “Semapeptide Gene” refers to nucleotide sequence of semapeptide or Arg34GLP-1 (9-37). The nucleotide sequence of semapeptide (nucleotide sequence) is: GAG GGC ACC TTC ACC AGC GAC GTT AGC AGC TAT CTG GAG GGC CAG GCG GCG AAA GAA TTT ATT GCG TGG CTG GTT CGT GGT CGT GGC.

The term “fermentation”, as used herein, is intended to refer to processes involving the production of recombinant protein products.

The term “room temperature” as used herein refers to the temperatures of the thing close to or same as that of the space, e.g., the room or fume hood, in which the thing is located’. Typically, room temperature can be from about 20° C. to about 30° C., or about 22° C. to about 27° C., or about 25° C.

The reactions of the processes described herein can be carried out in air or under an inert atmosphere. Typically, reactions containing reagents or products that are substantially reactive with air can be carried out using air sensitive synthetic techniques that are well known to the person skilled in art.

The term “Aib” amino acid as used herein refers to 2-Aminoisobutyric acid, or α-aminoisobutyric acid or α-methylalanine or 2-methylalanine.

The term “Boc” as used herein refers to tert-butyloxycarbonyl. Boc is used herein as protecting group of amine groups of Histidine (His) amino acid.

Although the exemplified procedures herein illustrate the practice of the present invention in some of its embodiments, the procedures should not be construed as limiting the scope of the invention. Modifications from consideration of the specification and examples within the ambit of current scientific knowledge will be apparent to one skilled in the art.

### EXAMPLE 1

Cloning and Expression of semapeptide (nucleotide sequence)-Ubiquitin fusion construct in E. coli.

Step a. Cloning of synthetic gene construct of semapeptide (nucleotide sequence) with Ubiquitin fusion tag.

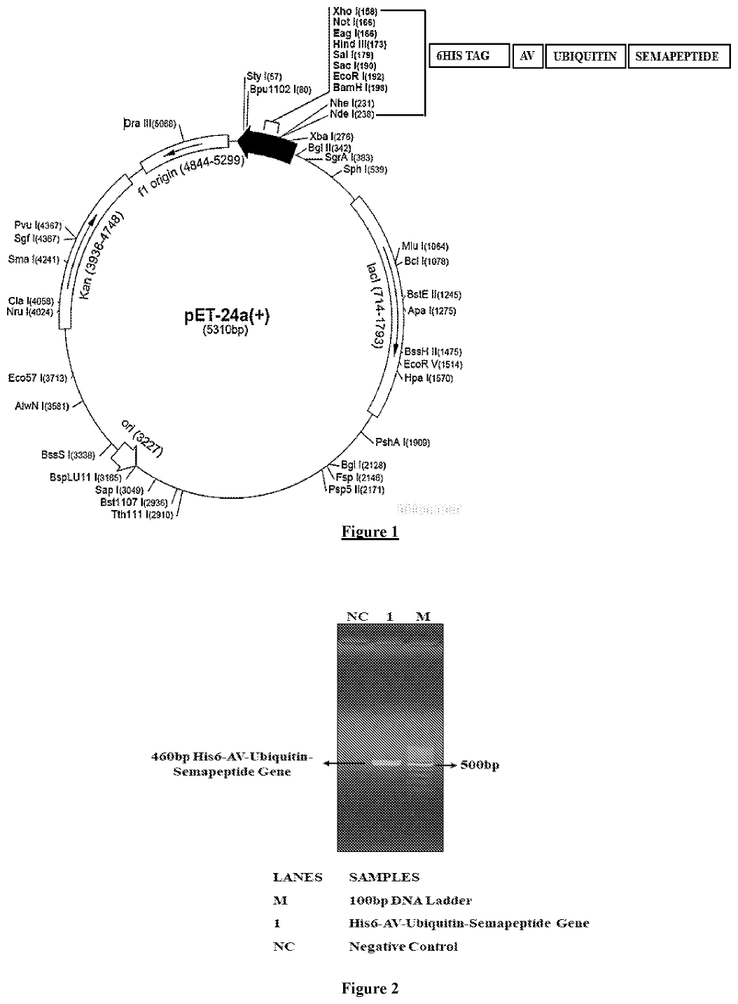

The His6-AV-Ubiquitin-semapeptide (nucleotide sequence) fusion protein encoding gene was amplified using forward primer (Primer 3) and T7 reverse primer (Universal primer) and pET24-Arg34GLP-1 (9-37) without 6His Tag single copy (recombinant plasmid generated from combination of Primer 1 & Primer 2) as a template. The Q5 High Fidelity DNA polymerase was used for the PCR amplification. The desired monocistronic (single copy) of His6-AV-Ubiquitin- semapeptide (nucleotide sequence)-encoding gene PCR product of ˜320bp was obtained as evident by 1% (w/v) Agarose gel electrophoresis (FIG. 1). The primers sequence is reiterated below:

| Primer | | Protein |
|---|---|---|
| no | Nucleotide Sequence | Sequence |
| Primer 1 | GATATCCATATGCAGATTTTCGT | HMQIFVKT |
| | GAAAACC | |
| Primer 2 | GCTAGCCTCGAGTTATTAGCCAC | LVRGRG |
| | GACCACGAACCAG | |
| Primer 3 | GACGTCATATGCACCACCACCAC | HMHHHHHHAVMQ |
| | CACCACGCGGTGATGCAGATTTT | IFVK |
| | CGTGAAA | |

Step b. Sub-cloning & Characterization

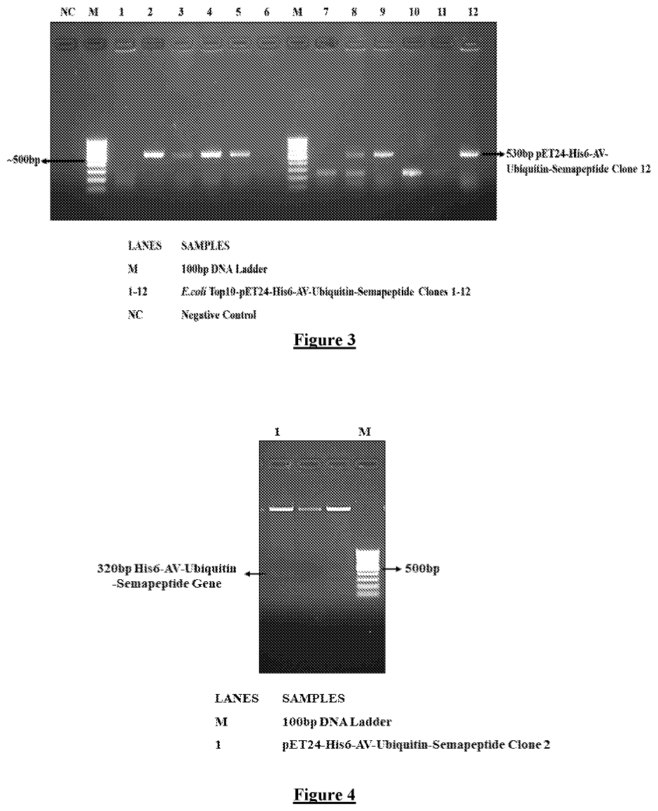

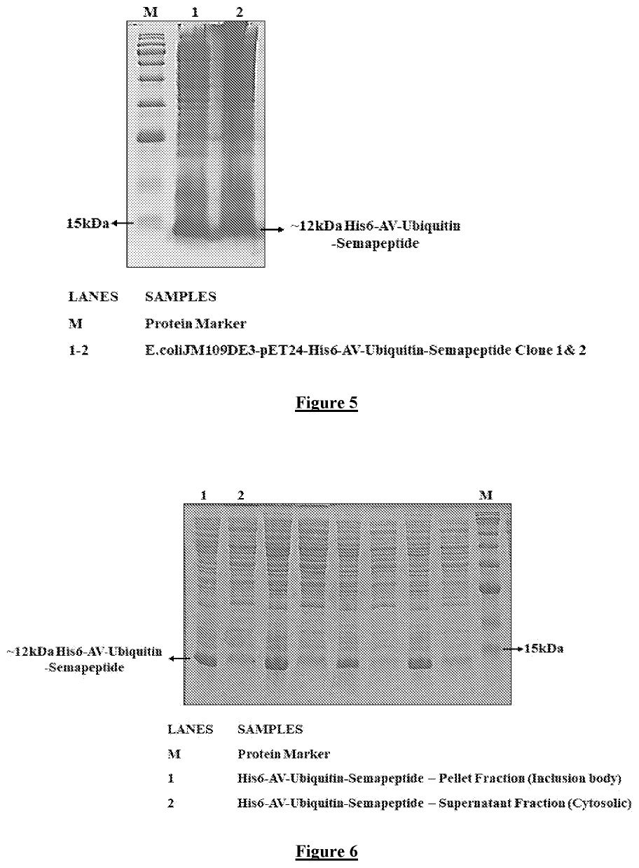

The His6-AV-Ubiquitin-semapeptide (Nucleotide sequence) encoding gene PCR product was gel purified. The pET24 plasmid vector was isolated from overnight grown E. coli Top10-pET24 culture. The gel purified His6-AV-Ubiquitin-semapeptide (nucleotide sequence) encoding gene PCR product and pET 24 plasmid vector was restriction digested (double digestion) with NdeI(NEB +XhoI (NEB) at 37° C. The digested Sema (N-2)-AV fusion protein encoding gene PCR product and pET24 plasmid vector was gel purified. The gel purified digested His6-AV-Ubiquitin- semapeptide (nucleotide sequence) encoding gene PCR product was ligated into Ndel and Xhol sites of pET24 plasmid vector using Quick ligation kit (NEB). The ligation mix was transformed into E. coli Top10 cells by heat shock method. The transformed cells were grown on LB agar plate+Kanamycin at 37° C. The transformed colonies were screened by colony PCR using T7 primers for the positive clones. DNA polymerase was used for the PCR amplification (FIG. 2). The PCR positive clones were further confirmed by restriction digestion analysis of recombinant His6-AV-Ubiquitin-Semapeptide (nucleotide sequence)-pET24 plasmid vectors using NdeI (NEB)+XhoI (NEB) at 37° C. The release of His6-AV-Ubiquitin-semapeptide (nucleotide sequence) encoding gene insert was observed on 1% (w/v) Agarose gel electrophoresis (FIG. 3).

Step c: Expression studies of His6-AV-Ubiquitin-Arg34GLP-1 (9-37)) Peptide in E. coli JM109DE3 expression host

The recombinant His6-AV-Ubiquitin-Semapeptide (nucleotide sequence)-pET24 plasmid vector was transformed into E. coli JM109DE3 cells by heat shock method. The transformed cells were grown on LB agar plate +Kanamycin at 37° C. The transformed colonies of E.coli JM109DE3 His6-AV-Ubiquitin-Semapeptide (nucleotide sequence)-pET24 expression clones were screened for the His6-AV-Ubiquitin-Semapeptide fusion protein expression. The His6-AV-Ubiquitin-semapeptide expression in E. coli JM109DE3 expression host was found to be good and insoluble (inclusion body) (FIGS. 4 & 5). The cell culture was mixed with 50% sterile glycerol and obtain aliquot having recombinant clone of E. coli containing His6-AV-Ubiquitin-Semapeptide (nucleotide sequence)-pET24 peptide. The obtained aliquot poured into Cryovials and stored at -80° C.

### Example 2

### Fermentation Process

Step a): Preparation of Pre-Seed:

100 ml Pre-seed medium is added in 500 ml Erlenmeyer flasks by using raw materials as mentioned in Table 1 was prepared. And pH of pre seed medium was adjusted to 7.2.

TABLE 1
Pre-seed Media Composition

| Sl. No | Media component |
|---|---|
| 1 | Yeast Extract - R |
| 2 | Sodium Chloride |

Kanamycin sulphate solution was added in Erlenmeyer flasks containing pre seed medium. Pre-seed culture was made by inoculating 0.2% (v/v) working cell bank of E. coli containing His6-AV-Ubiquitin-semapeptide (nucleotide sequence)-pET peptide in to Erlenmeyer flask having media composition as given in Table 1. The pre-seed culture was incubated in an orbital shaker at 37±1.0° C. for 9-12 hours.

Step b): Preparation of seed medium in seed fermenter:

500 ml of seed medium was prepared in 2L Erlenmeyer flasks by using raw materials as mentioned in Table 2. The pH of seed medium was adjusted to 7.2.

TABLE 2
Seed Media Composition

| Sl. No | Media component |
|---|---|
| 1 | Yeast extract-R |
| 2 | NaCl |
| 3 | K2HPO4 |
| 4 | MgSO4•7H2O |
| 5 | Glucose |

Kanamycin sulphate solution was added in sterile Erlenmeyer flasks containing seed medium. Seed was made by inoculating 2% (v/v) Pre-seed culture to seed fermenter having media composition as shown in Table 2. The seed was grown for 8-10 hrs.

Step c) Preparation of culture media for Production Tank Fermenter: The production tank fermentation medium of 20 liters was prepared according to components mentioned in Table.3 and sterilized. The fermentation medium was poured in production tank fermenter and sterilized.

TABLE 3
Production Media Composition

| Sl. No | Media Component |
|---|---|
| 1 | Potassium dihydrogen phosphate |
| 2 | Diammonium hydrogen phosphate |
| 3 | Yeast extract |
| 4 | Soypeptone |
| 5 | Antifoam |
| 6 | Citric acid anhydrous |
| 7 | Calcium chloride dihydrate |
| 8 | Thiamine hydrochloride |
| 9 | EDTA sodium salt |
| 10 | Cobaltous chloride hexhydrate |
| 11 | Manganase chloride tetrahydrate |
| 12 | Copper sulphate pentahydrate |
| 13 | Boric acid |
| 14 | Sodium molybdate dihydrate |
| 15 | Zinc sulphate heptahydrate |
| 16 | Ferrous sulphate heptahydrate |
| 17 | Ammonium sulphate |
| Sl. No. | Feed Media Component |
| 1 | Magnesium sulphate heptahydrate |
| 2 | Glucose |

| Sl. No | Feed Media Component |
|---|---|
| 1 | Yeast Extract |
| 2 | Ammonium Sulphate |
| 3 | Glucose |
| 4 | Magnesium Sulphate |

Step d) Preparation of Supplement component of fermentation: 1) Glucose and magnesium sulphate heptahydrate was prepared in 1-liter RO water. 2) Liquor Ammonium (12.5%) was prepared in RO water of about 1500 ml. 3) Antiform 204 was prepared in RO water of about 500 ml.

Feed Preparation: Feed medium of 12 liters for fermentation was prepared according to the components of Table 3 and sterilized.

Step e) Procedure for fermentation process: After temperature reaches to set point 37° C. of the production tank medium, glucose and magnesium sulphate heptahydrate aseptically was added in bio vessel and adjusted to pH 7.0. Further, inoculate mature seed (1L) from seed fermenter into production tank and maintained pH 7.0±0.2. Feed was added to fermenter on rise of pH and DO. Further, tank medium was induced by 1 M IPTG solution. All other parameters were maintained for 6-8 hrs after induction. After completion of fermentation, the temperature of broth was decreased to 10-15° C., centrifuged, collected cell mass and stored at −80° C.

Step f) Homogenization and clarification by Centrifuge:

150 g of cell pellet stored in -80° C. freezer was subjected to homogenization for cell lysis using 1.5L Lysis buffer (50 mM Tris-HCl+8 M Urea, pH 8.0±0.1) for 5-6 cycles. The lysate solution was clarified by centrifugation. The clarified supernatant fraction containing His6-AV-Ubiquitin-semapeptide was transferred to a beaker to load onto Ni-NTA resins while the pellet fraction containing cell debris was discarded.

### Example 3

### Purification of Fusion Protein Using Nickel NTA Chromatography

Permeate having His6-AV-Ubiquitin-Arg34GLP-1 (9-37) peptide was loaded on to the affinity chromatography (Ni-NTA) which was equilibrated with urea buffer (8 M urea+50 mM Tris, pH 8.0). After loading, matrix washed with same urea buffer and started eluting the impurities with elution buffer containing 20 mM Imidazole followed by 250 mM Imidazole to give elute having pure His6-AV-Ubiquitin-semapeptide.
Equilibration buffer: 50 mM Tris-HCl
   8 M Urea, pH 8.0±0.1
Sample loading: Clarified supernatant fraction 1400 ml at room temperature
Plain wash buffer: 50 mM Tris-HCl, pH 8.0±0.1;
Wash buffer: 50 mM Tris-HCl
   20 mM Immidazole, pH 8.0±0.1;
Elution Buffer: 50 mM Tris-HCl
   250 mM Immidazole, pH 8.0±0.1;

### Example 4

### YUH1/UHE Enzymatic Cleavage of Ubiquitin Tag from His6-AV-Ubiquitin-Semapeptide to Obtain Semapeptide

The His6-AV-Ubiquitin-semapeptide subjected to YUH1/UHE enzymatic cleavage to remove the Ubiquitin tag from His6-AV-Ubiquitin-Semapeptide to yield Semapeptide. The cleavage sample was analyzed by HPLC to check the cleavage percentage and Semapeptide quantity. The cleavage percentage was found to be (85) % whereas the Semapeptide quantity was found to be 930 mg/L.

### Example 5

### Isoelectric Point (Pi) Precipitation of Semapeptide

Semapeptide present in cleavage sample was subjected to isoelectric point (pI) precipitation. The pH was adjusted to pI value of Semapeptide viz pH 4.8±0.1. The sample was incubated at 2-8° C. refrigerator for precipitation. The pI precipitation sample was centrifuged. The supernatant fraction was discarded whereas the pellet fraction containing crude Semapeptide was stored at −20° C.

1. A process for producing Arg³⁴GLP-1 (9-37) peptide comprising the steps of:
a) culturing a host cell comprising a nucleotide sequence encoding of Formula (II) under suitable conditions for expression,
Insoluble tag-Fusion tag-Arg³⁴GLP-1 (9-37) Formula (II)
wherein, insoluble tag is a nucleotide sequence of Alanine-Valine; and
b) recovering the expressed Arg³⁴GLP-1 (9-37) peptide.

2. The process according to claim 1, wherein the said insoluble tag is optionally linked to a nucleotide sequence encoding affinity tag of Formula (III),
Affinity Tag-Insoluble tag-Fusion tag-Arg³⁴GLP-1 (9-37) Formula (III).

3. The process according to claim 2, wherein the affinity tag is selected from Polyarginine-tag (Arg-tag), Polyhistidine-tag (His-tag), S-tag, SBP-tag (streptavidinbinding peptide), Maltose binding protein and chitin binding domain (CBD).

4. The process according to claim 1, wherein multiple copies of nucleotide sequence encoding a formula (II) cloned together for expression.

5. The process according to claim 1, which further comprises increase in accumulation of resulting Arg³⁴GLP-1 (9-37) peptide by fermentation.

6. The process according to claim 5, which comprises:
a) inducing transformant prokaryotic cells which comprise of expression vector and a nucleotide sequence encoding Formula (II) or Formula (III) in fermentation culture medium;
b) culturing the transformant prokaryotic cells under condition suitable for accumulation of Arg³⁴GLP-1 (9-37) peptide;
c) recovering the expressed Arg³⁴GLP-1 (9-37) peptide, d) optionally, purifying the expressed Arg³⁴GLP-1 (9-37) peptide;
e) enzymatically cleaving the fusion tag from the expressed Arg³⁴GLP-1 (9-37) peptide; and
f) recovering Arg³⁴GLP-1 (9-37) peptide; and
g) optionally, purifying Arg³⁴GLP-1 (9-37) peptide.

7. A process for the preparation of semaglutide, comprising the steps of:
a) culturing a host cell comprising a nucleotide sequence encoding a Formula (II) or Formula (III) under suitable conditions for expression,
Insoluble tag-Fusion tag-- Arg³⁴GLP-1 (9-37) Formula (II)
Affinity Tag-Insoluble tag-Fusion tag-- Arg³⁴GLP-1 (9-37) Formula (III)
wherein, insoluble tag is nucleotide sequence of Alanine-Valine;
b) recovering the expressed Arg³⁴GLP-1 (9-37) peptide;
c) acylating an epsilon amino group one lysine residue in the expressed Arg³⁴GLP-1 (9-37) with an acylating agent of Formula (V), which is optionally activated, 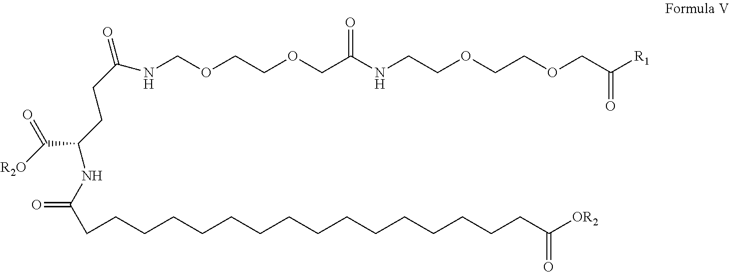
d) coupling optionally protected Aib and His amino acid, and
e) isolating semaglutide.

8. A conjugate of Formula IV,
His6 Tag-AV-Ubiquitin-Arg³⁴GLP-1 (9-37) (Nucleotide Sequence) Formula (IV).

9. The process according to claim 2, which further comprises increase in accumulation of resulting Arg³⁴GLP-1 (9-37) peptide by fermentation.
